(19) United States
(12) Patent Application Publication
(10) Pub. No.: US 2004/0005610 A1
(43) Pub. Date: Jan. 8, 2004

(54) Computational determination of alternative splicing

(76) Inventor: Rangarajan Sampath, San Diego, CA (US)

Correspondence Address: COZEN O'CONNOR, P.C., 1900 MARKET STREET, PHILADELPHIA, PA, 19103-3508, US

(21) Appl. No.: 10/434,564

(22) Filed: May 9, 2003

Related U.S. Application Data
(60) Provisional application No. 60/379,229, filed on May 9, 2002.

(51) Int. Cl. C12Q 1/68; G06F 19/00; G01N 33/48; G01N 33/50

(52) U.S. Cl. 435/6; 702/20

(57) ABSTRACT

The present invention provides methods of determining the presence of an alternative transcript form of a nucleic acid molecule. An mRNA sequence is mapped onto a corresponding genomic DNA sequence to reveal at least one mRNA exon fragment. An expressed sequence tag database is interrogated for expressed sequence tags similar to the genomic DNA sequence to generate a collection of expressed sequence tags. The expressed sequence tags in the collection are clustered. The presence of two or more clusters of expressed sequence tags indicates the presence of an alternative transcript form of the nucleic acid molecule.

### CROSS-REFERENCE TO RELATED APPLICATIONS

This application claims priority to U.S. provisional application Serial No. 60/379,229 filed May 9, 2002, which is incorporated herein by reference in its entirety.

### FIELD OF THE INVENTION

The present invention is directed, in part, to methods for determining the presence of alternative splice forms. In particular, the present invention allows association of a particular alternative transcript form to a particular medical condition via biological information associated with expressed sequence tags.

### BACKGROUND OF THE INVENTION

During the early stages of molecular biology and genetics it was thought that one gene encoded one protein. But as research continued it was discovered that this dogma was incorrect. Rather, as genes are transcribed and translated into proteins the DNA that encodes for a specific gene often times goes through a multistep process. The first step of this process is the copying of the DNA into RNA using the transcription machinery of the cell. The RNA that is initially produced in higher organisms, and especially in humans, is a combination of exons and introns. This form of RNA is normally not translated into the protein product; rather, this RNA is transformed into messenger RNA (mRNA), which is then translated into the final protein product.

The step(s) of transforming RNA to mRNA involves splicing the exons together and excluding the introns to create a transcript that encodes for a protein. An implication of splicing is that multiple or alternative forms of mRNA can be produced for a single gene producing different or alternative forms of the protein after translation. Additionally, these alternative transcript forms can have different properties that affect mRNA stability or translational regulations and, therefore, regulating protein production. In addition, these proteins may have different functions and roles within the cell. Thus, splicing plays an important role in the bioactivity of the cell and the organism.

Different splice forms can also be important in diseases. It is thought that alternative transcript forms of specific genes play a role in tumor progression in many forms of cancer. For example, over 40 different transcripts have been identified for the mdm2 gene that may be important in tumor survival and tumor progression. These different transcript forms make attacking multiple forms of cancer very difficult due to the heterogeneous nature of cancer.

There appear to be RNA sequences and structures that distinguish cancer and normal cells which are referred to herein as cancer signatures in RNA. Cancer signatures may be essential to the cancer phenotype or they may be non-essential changes acquired during the development of cancer. The cancer signatures can provide the recognition element to selectively identify and destroy cells.

Expressed sequence tags (ESTs) are short nucleotide sequences produced from randomly selected cDNA clones. They can be used for gene identification, creation of gene catalogs, as well as production of transcript profiles. Complete human EST database (dbEST) and cancer specific (CGAP-EST) databases are available from public resources (NCBI). From our analysis of ESTs, it has been found that mRNA transcripts are much more heterogeneous than anyone had anticipated. Many genes have as many as 10-20 alternative transcript forms that, in some cases, have been associated with a cancer phenotype. For example, in cancerous cells, transcription of the mdm2 gene is initiated at a distinct site not used in normal cells. Landers et al., Cancer Res., 1997, 57, 3562-3568. In the Bcl-x mRNA, alternatively spliced forms of the transcript result in dramatically different cell behavior and sensitivity to chemotherapeutic drugs. Kuhl et al., Br. J. Cancer, 1997, 75, 268-274.

We propose that a very rich source of RNA cancer signatures may be hidden in mRNA alternative transcript forms. Alternative transcript forms, as used herein, are mRNAs derived from the same gene, but containing different sequences and structures. Alternative transcript forms originate from, for example, alternative initiation of transcription, alternative splicing, alternative 3′-end processing, or a combination of these mechanisms.

Studying 160,000 EST sequences, Gautheret et al. have shown that from 20-40% of the transcripts have two or more different 3′-ends. Gautheret et al., Genome Res., 1998, 8, 524-530. Other investigators have shown that certain classes of mRNAs are alternatively 3′-end processed in a tissue-specific or developmentally specific pattern (Edwalds-Gilbert et al., Nucleic Acids Res., 1997, 25, 2547-2561) and, in some cases, this has been correlated with cancer. For example, the mss4 transcript was recently shown to have alternative 3′-end processing in pancreatic cancer. Muller-Pillasch et al., Genomics, 1997, 46, 389-396. Another example of differential translational regulation due to tissue-specific alternative polyadenylation is the 15-lipoxygenase mRNA. Thiele et al., Nucleic Acids Res., 1999, 27, 1828-1836. Non-erythroid tissues (heart, lung, etc.) express a long form of 15-LOX that is in a translationally non-repressed state, probably due to the binding of specific proteins that do not bind the short form expressed in reticulocytes. Alternative 3′-end formation does not change the protein composition, but can dramatically influence message stability and regulate translation by including or excluding regulatory sequences in the mRNA transcript.

A very important consequence of alternative transcript forms for cancer recognition is the unique shapes that they create. In contrast to the regular helical nature of DNA, RNA strands form intricate stems, loops, and bulges, which are arranged into three-dimensional shapes that rival proteins in their complexity. Alternative transcript forms can produce different shapes in several ways. First, with alternative transcription initiation or 3′-end formation, there are unique sequences in mRNAs that do not appear at all in the normal mRNA. These sequences, in turn, will fold into unique structures within themselves and with the adjacent RNA. Second, each alternative splicing event produces a unique junction, in which the adjacent RNA on each side of the junction will re-arrange into a new three-dimensional shape. It is important to distinguish the concepts of alternative transcript forms from cancer-specific expression of transcripts. Many investigators are pursuing transcripts and proteins that are expressed at different levels in cancer versus normal cells. Indeed, 500 transcripts have been reported to be expressed at significantly different levels (15-fold on average) in normal versus gastrointestinal tumor cells. Zhang et al., Science, 1997, 276, 1268-1272. Cancer-specific transcripts provide a useful set of molecular targets for the technology that proposed herein. The greater opportunity, however, to find useful cancer signatures may be in alternative transcript forms. Because there may be 2-20 different forms of every transcript and 10-20,000 genes expressed in any given cell, the opportunity to find cancer-specific alternative transcript forms may be much greater than for cancer-specific transcripts.

Whether the origin of the cancer signature comes from cancer-specific transcripts or cancer-specific transcript forms, it is not required that the cancer-specific differences in mRNA be responsible for cancer phenotype for the technology proposed herein. It is not even important that we know what they do. The important point is that they are present in cancer cells and can therefore be used to mark them for destruction.

Although alternative transcripts have been identified in the past, there is a need for a faster, cheaper and better way to identify alternative transcripts of any gene. Thus, there is a long-felt need for methods to identify alternative transcripts. There is also a long-felt need for methods of identifying specific alternative transcripts that are associated with medical conditions, such as diseased cells including, but not limited to, cancerous cells. The present invention fulfills these needs as well as others.

### SUMMARY OF THE INVENTION

The present invention provides methods of determining the presence of an alternative transcript form of a nucleic acid molecule comprising mapping an mRNA sequence onto a corresponding genomic DNA sequence to reveal at least one mRNA exon fragment, interrogating an expressed sequence tag database for expressed sequence tags similar to the genomic DNA sequence to generate a collection of expressed sequence tags, and clustering expressed sequence tags in the collection, wherein the presence of two or more clusters of expressed sequence tags indicates the presence of an alternative transcript form of the nucleic acid molecule.

### BRIEF DESCRIPTION OF THE DRAWINGS

FIG. 1 shows a map alternative splicing using EST analysis of an 8 kb region on a BAC Clone (marked as “Query”); 5 exons were present in the longest transcript (a); a splice variant with an alternate 5′ end (b); and another splice variant missing exon 3 (c).

### DESCRIPTION OF EMBODIMENTS

The present invention provides methods of determining the presence of an alternative transcript form of a nucleic acid molecule. The present invention also provides methods of identifying alternative transcript forms. The alternative transcript forms can be associated with biological information, including medical conditions, such as cancer. Therefore, the discovery of new alternative transcript forms by the present methods can lead to new targets and approaches for therapy and/or prevention of disease conditions.

In some embodiments, the present invention comprises mapping an mRNA sequence onto a corresponding genomic DNA sequence to reveal at least one mRNA exon fragment. The mRNA sequence can be a complete mRNA sequence or a partial mRNA sequence. The mRNA sequence can be a proprietary sequence or within public knowledge. In addition, the mRNA sequence can be derived from a cloned sequence or can be assembled from fragments of mRNA sequences. In some embodiments, the mRNA sequence is known to be involved in a disease condition.

The corresponding genomic DNA sequence is the genomic DNA sequence that contains the gene from which the particular mRNA derives. Numerous depositories of genomic DNA sequences are known to those skilled in the art and can be sought as desired. The genomic DNA sequence can be from an animal, mammal, or human. In some instances, the mRNA sequence will have been produced from a sequence that contains no introns. In this case, once the mRNA sequence is mapped to the genomic DNA sequence, only one mRNA exon fragment will be revealed (i.e., the mRNA sequence contains only one exon). In other instances, the mRNA sequence will have been produced from a sequence that contains one or more introns. In this case, once the mRNA sequence is mapped to the genomic DNA sequence, more than one mRNA exon fragment will be revealed (i.e., the mRNA sequence contains more than one exon). Mapping to reveal mRNA fragments can be accomplished by, for example, aligning the mRNA sequence with the corresponding genomic DNA sequence. Such mapping can be carried out by, for example, using a basic local alignment search tool such as BLAST (Altschul et al., J. Mol. Biol., 1990, 215, 403-410), Fasta (Pearson et al., Proc. Natl. Acad. Sci. USA, 1988, 85, 2444-2448), or Smith-Waterman (Smith et al., J. Mol. Biol., 1981, 147, 195-197).

In some embodiments of the invention, either terminus or both termini of the corresponding genomic DNA sequence corresponding to the 5′-termini of the 5′-most mRNA exon fragment and the 3′-termini of the 3′-most mRNA exon fragment can optionally be extended to generate an extended genomic DNA sequence. A particular mRNA sequence, for example, may map to its corresponding genomic DNA sequence and reveal three mRNA exon fragments (e.g., including a 5′-most mRNA exon fragment, a middle fragment, and a 3′-most mRNA exon fragment). If the genomic DNA sequence is not extended, the 5′-terminus of the genomic DNA sequence correlates with the 5′-terminus end of the 5′-most mRNA exon fragment and the 3′-terminus of the genomic DNA sequence correlates with the 3′-terminus of the 3′-most mRNA exon fragment. In instances where a terminus of the genomic DNA sequence is extended, the genomic DNA sequence can be extended between about 1 kilobase to about 5 kilobases on each terminus. As used herein, “about” means ±10% of the value it modifies.

In some embodiments of the invention, the genomic DNA sequence or the extended genomic DNA sequence can be used to interrogate an expressed sequence tag database for expressed sequence tags similar to the genomic DNA sequence or extended genomic DNA sequence, as the case may be, to generate a collection of expressed sequence tags. The interrogation can be carried out by, for example, using a basic local alignment search tool such as those described above. Expressed sequence tag databases including, for example, dbEST and CGAP-EST, are well known and available to one skilled in the art. The CGAP-EST database comprises cancer-specific expressed sequence tags. Any database, however, containing any number of expressed sequence tags can be used. Further, every expressed sequence tag present in the database can be used in the interrogation or, alternately, less than every expressed sequence tag present in the database can be used in the interrogation.

Because of the variability of data available from the various expressed sequence tag databases (i.e., the underlying expressed sequence tag sequence itself and the associations with tissues of origin and/or disease states), in some embodiments of the invention additional steps can be taken. Variability in expressed sequence tag sequences can be addressed, for example, by using EST trace data available from the Wash-U sequencing center file transfer program (genome.wustl.edu/pub/gscl/est). Trace viewers such as “ted” available from the same site, or JAVA based tools (Parsons et al., i Genome Res., 1999, 9, 277-281) can be used to view and analyze individual traces. Variability in the associations with tissues of origin and/or disease states can arise because there can be inconsistency in the preparation of the libraries, and a significant bias towards certain tissues of origin for many of the libraries. In addition, some forms of cancer are represented more than others. Thus, in some embodiments of the invention, pooled libraries for each source tissue (e.g. lung, fetal, brain, etc.), and for each known pathological state of the source (e.g. normal, cancer, etc.) are used. An evaluation of the statistical significance of an observed bias is possible through the use of, for example, Fisher's 2×2 exact test. This computes the probability of a given 2×2 occurrence table for two independent categories. Here, one category is the source library and the other the mRNA form. Fisher's test can be applied to every pair of such pooled libraries, for any given gene, to track possible significance in the observed variation in pattern of tissue expression. The calculations can be performed at the web interface provided by Oyvind Langsrud at www.matforsk.no/ola/fisher.htm.

In some embodiments, the expressed sequence tag database can optionally be screened prior to interrogating to remove vector sequences, recognized repetitive elements (e.g., Alu, LINE, LTRs, etc.), or low-complexity regions, or any combination thereof from the database. In addition, interrogation of an expressed sequence tag database can include mapping of each interrogated expressed sequence tag to the genomic DNA sequence or extended genomic DNA sequence. Thus, a result of the interrogation is a collection of expressed sequence tags that can be mapped to the genomic DNA sequence or extended genomic DNA sequence.

After interrogation of one or more expressed sequence tag databases, the collection of expressed sequence tags that map to the genomic DNA sequence or extended genomic DNA sequence may comprise one or more expressed sequence tags that extend beyond the 5′-terminus of the genomic DNA sequence or the extended genomic DNA sequence or that extend beyond the 3′-terminus of the genomic DNA sequence or extended genomic DNA sequence. For example, the collection of expressed sequence tags may comprise one expressed sequence tag that comprises a 3′ portion that overlaps with the 5′-most portion of the genomic DNA sequence or the extended genomic DNA sequence and, thus, comprises a sequence portion (i.e., the 5′-terminus portion of the expressed sequence tag) that dangles from the genomic DNA sequence or the extended genomic DNA sequence when aligned thereto. In such cases, the relevant terminus of the genomic DNA sequence or extended genomic sequence can optionally be extended to generate an extended genomic DNA sequence or a further extended genomic DNA sequence, respectively, in order to generate a more complete collection of expressed sequence tags.

The genomic DNA sequence or extended genomic DNA sequence can be extended between about 1 kilobase to about 5 kilobases on the 5′-terminus of the genomic DNA sequence or extended genomic DNA sequence, the 3′-terminus of the genomic DNA sequence or extended genomic DNA sequence, or both the 5′-terminus and 3′-terminus of the genomic DNA sequence or extended genomic DNA sequence. An expressed sequence tag database can then be interrogated using the newly created extended genomic DNA sequence or further extended genomic DNA sequence. This process can be iteratively carried out with further extensions of the particular genomic DNA sequence until no expressed sequence tag that extends beyond the 5′-terminus of the particular genomic DNA sequence or that extends beyond the 3′-terminus of the particular genomic DNA sequence is found (i.e., no more dangling expressed sequence tags are found).

Once an expressed sequence tag database has been interrogated and a collection of expressed sequence tags has been generated, all or part of the collection of expressed sequence tags can be clustered. Each cluster of expressed sequence tags that is generated represents an alternative transcript form of the nucleic acid molecule. The presence of two or more clusters indicates the presence of at least one alternative transcript form. In addition, the expressed sequence tags of at least one cluster can be assembled to generate an alternative transcript form of the nucleic acid molecule. Generation of alternative transcript forms can be carried out by using commercially available programs. Contig-building programs, for example, can be used to assemble expressed sequence tags into alternative transcript forms. The CAP program, for example, can be used to assemble the expressed sequence tags within a particular cluster to generate an alternative transcript form. Huang, Genomics, 1992, 14, 18-25. Assembly of the expressed sequence tags within a particular cluster allows one skilled in the art to identify an alternative transcript form.

In some embodiments of the invention, expressed sequence tags that are similar to an extended genomic DNA sequence (e.g., the expressed sequence tag aligns with the genomic DNA sequence) and which do not overlap with any mRNA exon fragment can be removed from the collection of expressed sequence tags. In such embodiments, all or a part of the collection containing the remaining expressed sequence tags can be assembled to generate at least one alternative transcript form of the nucleic acid molecule. At least one of the removed expressed sequence tags can then be interrogated using at least one alternative transcript form that is generated.

Clustering is carried out for an expressed sequence tag in the collection. One, some, or all of the expressed sequenced tags in the collection can be clustered, as described below. For purposes of clustering, the mapped mRNA sequence comprising at least one mRNA exon fragment forms a first cluster. The first cluster can comprise as many mRNA exon fragments as are present in the particular mRNA sequence from which they are derived. For example, an mRNA sequence derived from a sequence having three exons and two introns can have three mRNA exon fragments when mapped to a particular genomic DNA sequence.

In an initial clustering, an expressed sequence tag in the collection is compared to the first cluster. If the expressed sequence tag overlaps any mRNA exon fragment within the first cluster (i.e., if the expressed sequence tag has at least one dangling base compared to any mRNA exon fragment with which it overlaps), then the expressed sequence tag forms a second cluster. Determining whether a particular expressed sequence tag overlaps any mRNA exon fragment within the first cluster can be conveniently carried out by aligning the expressed sequence tag with the mRNA exon fragment(s) present in the first cluster. If, however, the expressed sequence tag wholly resides within any mRNA exon fragment within the first cluster (i.e., if the expressed sequence has no dangling bases compared to any mRNA exon fragment to which it aligns), then the expressed sequence tag is added to the first cluster. Finally, if the expressed sequence tag does not overlap with any mRNA exon fragment in the first cluster, then the expressed sequence tag forms a second cluster. For example, a particular expressed sequence tag may align with a genomic DNA sequence and not align to any mRNA exon fragment. In this case, the expressed sequence tag forms a second cluster.

Subsequent rounds of clustering can be carried out on a plurality of expressed sequence tags remaining in the collection in either a sequential or concomitant fashion. Another expressed sequence tag can be compared to the first cluster of mRNA exon fragments or to the expressed sequence tag(s) present in the second cluster, if previously generated. The particular expressed sequence tag that is being analyzed is clustered into either a previously identified cluster (i.e., either the first cluster or second cluster, if previously identified) or is clustered into a new cluster. Such cluster determination is made by a set of clustering rules, such as those described below.

If the another expressed sequence tag wholly resides within any mRNA exon fragment of the first cluster or within any expressed sequence tag of the second cluster, then the another expressed sequence tag is added to either the first cluster or second cluster or both clusters. For example, if the another expressed sequence tag is entirely present within an mRNA exon fragment within the first cluster, then the another expressed sequence tag is added to the first cluster. Alternately, if the another expressed sequence tag is entirely present within an expressed sequence tag within the second cluster, then the another expressed sequence tag is added to the second cluster. In these cases, the another expressed sequence tag can be added to both the first and second clusters because the another expressed sequence tag is, in effect, redundant.

If the another expressed sequence tag overlaps with any mRNA exon fragment within the first cluster and does not overlap with an expressed sequence tag of the second cluster, then the another expressed sequence tag forms a third cluster. For example, if the another expressed sequence tag shares sequence alignment with an mRNA exon fragment within the first cluster but does not share sequence alignment with an expressed sequence tag within the second cluster, then the another expressed sequence tag forms a third cluster.

If the another expressed sequence tag does not overlap with any expressed sequence tag of the second cluster or with any mRNA exon fragment within the first cluster, then the another expressed sequence tag forms a third cluster. For example, the another expressed sequence tag may align only to the genomic DNA sequence and not to any member of any cluster. In this case, the another expressed sequence tag is placed into a third cluster.

If the another expressed sequence tag overlaps with an expressed sequence tag of the second cluster and which comprises no gap in the overlapping region when aligned to the expressed sequence tag within the second cluster, then the another expressed sequence tag is added to the second cluster. For example, the another expressed sequence tag may overlap with an expressed sequence tag of the second cluster. If the another expressed sequence tag does not comprise a gap in the overlapping region when it is aligned to the expressed sequence tag within the second cluster, then the another expressed sequence tag is added to the second cluster. A gap within an overlapping region is a gap in the another expressed sequence tag that is within the region that overlaps the expressed sequence tag in the second cluster.

Finally, if the another expressed sequence tag overlaps an expressed sequence tag of the second cluster and comprises a gap within the overlapping region when aligned to the expressed sequence tag within the second cluster, then the another expressed sequence tag forms a third cluster. For example, the another expressed sequence tag may overlap with an expressed sequence tag of the second cluster. If the another expressed sequence tag comprises a gap in the overlapping region when it is aligned to the expressed sequence tag within the second cluster, then the another expressed sequence tag is added to a third cluster. As stated above, a gap within an overlapping region is a gap in the another expressed sequence tag that is within the region that overlaps the expressed sequence tag in the second cluster.

In some embodiments, the clustering process can be iteratively carried out on a plurality of expressed sequence tags in the collection, in which each expressed sequence tag in the plurality is compared to the mRNA exon fragment(s) within the first cluster or compared to expressed sequence tag(s) of any subsequent cluster (e.g., second cluster or any subsequent cluster formed by previous iterations). In some embodiments, all expressed sequence tags in the collection can be clustered.

The fidelity of a particular cluster can be altered in some embodiments. For example, expressed sequence tags that align with an mRNA exon fragment or expressed sequence tag in any cluster which comprise ten or more mismatches at the terminal portion or comprise gapped internal alignments can be excluded from the cluster. A Fisher's exact test calculation can be performed to assess any significance. In addition, a particular expressed sequence tag that only appears once in the collection after interrogation of an expressed sequence tag database can be eliminated, as it may be indicative of an artifact.

Another aspect of the present invention is directed to determining the presence of alternate polyadenylation, which represents an important post-transcriptional regulatory process. Thus, the alternate transcript forms generated, as described above, can be further distinguished from one another based upon alternate polyadenylation. Gautheret et al. has developed ESTparser software that automatically aligns expressed sequence tags related to any particular mRNA and identifies alternate termination patterns. Using this tool in his study of a subset of an expressed sequence tag database (Washington University—Merck project), he has shown that about 20% of the human genes show alternate polyadenylation. Gautheret et al., Genome Res., 1998, 8, 524-530. Alternate 3′-terminus formation can dramatically influence message stability and regulate translation by including or excluding regulatory sequences in the mRNA transcript. In addition, unique regulatory elements that are characteristic of the various transcripts may be present in these regions and, thus, can be candidates for therapeutic targets.

As described above, each cluster represents an alternative transcript form. Any one or more clusters can be assembled, as described above, to generate alternative transcript form(s). In some embodiments, an expressed sequence tag used to assemble an alternative transcript form is associated with biological information. Thus, expressed sequence tags of each cluster can be assembled into alternative transcript forms and the biological information can be associated with the alternative transcript forms.

Biological information includes, but is not limited to, organ origin, tissue origin, disease state, developmental stage, or any combination thereof. Analysis of the alternative transcript forms can be carried out to determine correlation(s) between a particular alternative transcript form and a particular organ origin, tissue origin, disease state, or developmental stage, or any combination thereof. Thus, for example, a particular alternative transcript form can be correlated to a particular cancer in a particular tissue. Statistical evaluation techniques known to those skilled in the art can be used to make such correlations.

The alternate transcript forms can also be compared at high stringency against Genbank non-redundant database (NR) to assign putative function, as well to include all Genbank associated properties and annotations, if available. Additional information that may become available as a result of additional analyses, such as expression profiles, alternative splice sites, alternate start of transcription, presence of regulatory signals in the 3′ ends, etc. can be included as features of the annotation, thus adding significant value to genome sequence information.

The present invention also provides methods to automatically update the cluster database. Expressed sequence tags are constantly being generated and databases, especially the human EST sequence information within it (human dbEST), is growing at an explosive pace, doubling every six months or less. As it stands, there are close to 1.5 million human ESTs in the database as of Aug. 6, 1999. NCBI maintains a weekly update of this database. A batch process can be created that will automatically extract all new ESTs and assign them to their appropriate clusters, or create new clusters, as the data becomes available. While the initial clustering can be based on extensive pair-wise comparisons, placement of new sequences into appropriate clusters can be based on a “set theory” approach, as proposed by Krause et al. (Bioinformatics, 1998, 14, 430-438). In this approach, a sequence is identified as either belonging to a cluster or not with no heuristics, and is likely to speed up the process of clustering. In addition, a periodic update of annotations can be maintained, as they become available.

Another embodiment of the present invention includes using Java development tools, or the like, to make a web-based cluster viewing application, which will allow querying and sorting of clusters and contents of clusters. To view and query the contents of large clusters, Java based GUI applications can be developed that utilize the underlying database design. An easy to use yet powerful interface can be designed that can enable multi-level functionality. In addition, interfaces for viewing and querying cluster information can be provided. This interface can include a multiple sequence alignment viewer. These interfaces can also help create user-defined reports and can provide users with a customizable interface: properties such as specific color-coding, layout, etc. can be editable. Viewable properties can include all the features available in the cluster relational database, as well as those available through Gene Thesaurus links. Live links to public domain databases (CGAP, Genbank, TIGR-THC database, Unigene, Medline, etc.) can also be provided as appropriate. Java-based design can also eliminate hardware platform dependency, and can be easy to deploy.

The present invention is also directed to creating a database containing a plurality of alternative transcript forms. The database can be created by, for example, carrying out the methods described above. Further, the database can be updated with new alternative transcript forms. In addition, the alternative transcript forms within the database can be associated with biological information, as described above. To extract the most value from all the expressed sequence tag data, the expressed sequence tags and the alternate transcript forms assembled therefrom can be associate with relevant biological information. The database containing the same can be amenable to querying, filtering, and processing. Thus, the database can be a relational database that can be created from the cluster flat-file databases generated above, which can be used to track the virtual transcripts by cluster, tissue type, cancer disease state, and relative polyadenylation. In this manner, reliable annotations can be created in an automated manner that will guide design of further biological experiments to verify predicted gene structure, regulation and function.

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1

Splice Variants Within an 8 Kilobase Region of a BAC Clone

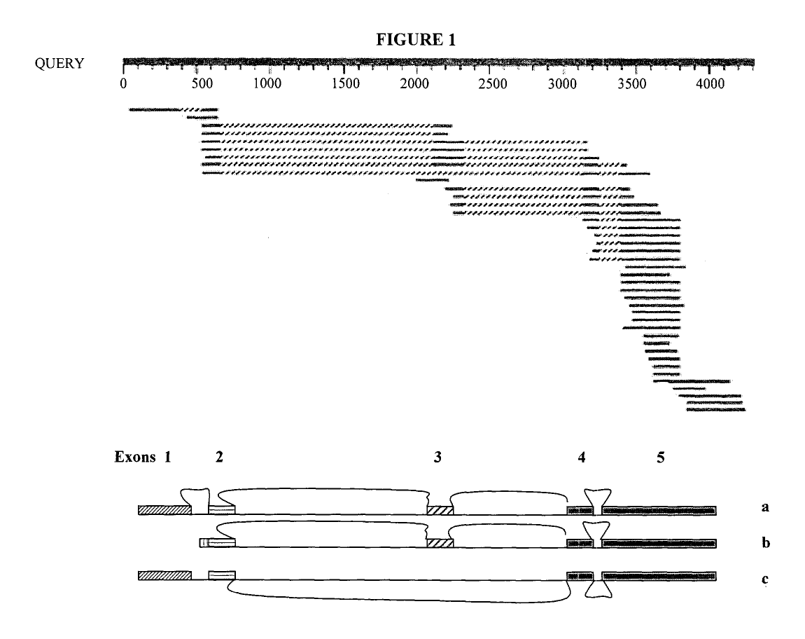

An 8 kb region on a BAC clone was used as the query for an initial BLAST search. Genbank annotations indicated that this region on the BAC clone did not correspond to any known gene. A collection of human ESTs was identified that showed significant homology to the query sequence. Using the clustering process described herein, the ESTs were divided into clusters, wherein each cluster represented a unique message. Full-length cDNA transcripts (i.e., contigs) representative of each cluster were created using overlapping ESTs to extend the 5′ and 3′ ends. Evidence for the presence of five exons in the longest transcript was found. Additionally, two splice variants, one with an alternate 5′ end and another missing exon 3 were also identified (see, FIG. 1).

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

What is claimed is:

1. A method of determining the presence of an alternative transcript form of a nucleic acid molecule comprising the steps of:
mapping an mRNA sequence onto a corresponding genomic DNA sequence to reveal at least one mRNA exon fragment;
interrogating an expressed sequence tag database for expressed sequence tags similar to the genomic DNA sequence to generate a collection of expressed sequence tags; and
clustering expressed sequence tags in the collection, wherein the presence of two or more clusters of expressed sequence tags indicates the presence of an alternative transcript form of the nucleic acid molecule.

2. The method of claim 1 further comprising extending each end of the corresponding genomic DNA sequence corresponding to the 3′-terminus of the 3′-most mRNA exon fragment and the 5′-terminus of the 5′-most mRNA exon fragment to generate an extended genomic DNA sequence.

3. The method of claim 2 wherein the genomic DNA sequence is extended between about 1 kilobase to about 5 kilobases on each end.

4. The method of claim 1 wherein the expressed sequence tag database is screened prior to interrogating to remove vector sequences, recognized repetitive elements, or low-complexity regions, or any combination thereof.

5. The method of claim 2 wherein if the collection of expressed sequence tags comprises an expressed sequence tag that extends beyond the 5′-terminus of the extended genomic DNA sequence or that extends beyond the 3′-terminus of the extended genomic DNA sequence, the genomic DNA sequence is further extended to generate a further extended genomic DNA sequence.

6. The method of claim 5 wherein the extended genomic DNA sequence is further extended between about 1 kilobase to about 5 kilobases on the 5′-terminus of the extended genomic DNA sequence, the 3′-terminus of the extended genomic DNA sequence, or both the 5′-terminus and 3′-terminus of the extended genomic DNA sequence.

7. The method of claim 5 wherein the further extended genomic DNA is iteratively extended until no expressed sequence tag extends beyond the 5′-terminus of the further extended genomic DNA sequence or beyond the 3′-terminus of the further extended genomic DNA sequence.

8. The method of claim 1 wherein the expressed sequence tags of at least one cluster are assembled to generate an alternative transcript form of the nucleic acid molecule.

9. The method of claim 5 wherein after interrogating, expressed sequence tags that are similar to the extended genomic DNA sequence and which do not overlap with any mRNA exon fragment are removed from the collection.

10. The method of claim 9 wherein the expressed sequence tags of at least one cluster are assembled to generate an alternative transcript form of the nucleic acid molecule, and at least one of the removed expressed sequence tags is interrogated using at least one alternative transcript form.

11. The method of claim 1 wherein the mapping is performed using a basic local alignment search tool.

12. The method of claim 1 wherein the interrogating is performed using a basic local alignment search tool.

13. The method of claim 1 wherein the mapped mRNA comprising at least one mRNA exon fragment forms a first cluster.

14. The method of claim 13 wherein at least one expressed sequence tag is compared to the first cluster, wherein:
if the expressed sequence tag overlaps any mRNA exon fragment within the first cluster, then the expressed sequence tag forms a second cluster;
if the expressed sequence tag wholly resides within any mRNA exon fragment within the first cluster, then the expressed sequence tag is added to the first cluster;
if the expressed sequence tag does not overlap with any mRNA exon fragment, then the expressed sequence tag forms a second cluster.

15. The method of claim 14 wherein another expressed sequence tag is compared to the first cluster or second cluster, wherein:
if the another expressed sequence tag wholly resides within any mRNA exon fragment of the first cluster or within any expressed sequence tag of the second cluster, then the another expressed sequence tag is added to either the first or second cluster or both clusters;
if the another expressed sequence tag overlaps with any mRNA exon fragment within the first cluster and does not overlap with an expressed sequence tag of the second cluster, then the another expressed sequence tag forms a third cluster;
if the another expressed sequence tag does not overlap with any expressed sequence tag of the second cluster or with any mRNA exon fragment within the first cluster, then the another expressed sequence tag forms a third cluster;
if the another expressed sequence tag overlaps with an expressed sequence tag of the second cluster and which comprises no gap in the overlapping region when aligned to the expressed sequence tag within the second cluster, then the another expressed sequence tag is added to the second cluster; or
if the another expressed sequence tag overlaps an expressed sequence tag of the second cluster and comprises a gap within the overlapping region when aligned to the expressed sequence tag within the second cluster, then the another expressed sequence tag forms a third cluster.

16. The method of claim 15 wherein each expressed sequence tag in the collection is compared to the mRNA exon fragment or fragments of the first cluster or to the expressed sequence tags of any subsequent cluster until all expressed sequence tags are clustered.

17. The method of claim 1 wherein an expressed sequence tag is associated with biological information.

18. The method of claim 17 wherein the expressed sequence tags of each cluster are assembled into alternative transcript forms and the biological information is associated with the alternative transcript forms.

19. The method of claim 17 wherein the biological information comprises organ origin, tissue origin, disease state, developmental stage, or any combination thereof.

20. The method of claim 8 further comprising creating a database containing a plurality of alternative transcript forms.

21. The method of claim 20 further comprising updating the database with new alternative transcript forms.

22. The method of claim 20 wherein the alternative transcript forms within the database are associated with biological information.
